SEQUENCE LISTING (3 sequences)

SEQ ID NO: 1 - DNA, 21 bp, synthetic construct
  misc_feature 1..21: note = target sequence in NMNAT1
  source 1..21: mol_type = other DNA
ggaaggaagaggaagtggact

SEQ ID NO: 2 - DNA, 19 bp, synthetic construct
  misc_feature 1..19: note = target sequence in NMNAT1
  source 1..19: mol_type = other DNA
acaggaaggtacacagttg

SEQ ID NO: 3 - DNA, 21 bp, synthetic construct
  misc_feature 1..21: note = target sequence in NMNAT1
  source 1..21: mol_type = other DNA
cgctacttggtaccagatctt

(19) United States
(12) Patent Application Publication
(10) Pub. No.: US 2025/0019708 A1
(43) Pub. Date: Jan. 16, 2025

(54) METHODS FOR THE TREATMENT OF HRD CANCER AND BRCA-ASSOCIATED CANCER

(71) Applicants: INSTITUT CURIE, Paris (FR); INSTITUT NATIONAL DE LA SANTE ET DE LA RECHERCHE MEDICALE (INSERM), Paris (FR)

(72) Inventors: Raphaël CECCALDI, Paris (FR); Daniele MUSIANI, Paris (FR); Yucel HATICE, Paris (FR)

(21) Appl. No.: 18/710,968

(22) PCT Filed: Nov. 17, 2022
(86) PCT No.: PCT/EP2022/082281

§ 371 Date: May 16, 2024

(30) Foreign Application Priority Data
Nov. 19, 2021 (EP) 21306616.0

(51) Int. Cl. C12N 15/113; A61K 31/55; A61K 33/243; A61P 35/00

(52) U.S. Cl. CPC C12N 15/1137; A61K 31/55; A61K 33/243; A61P 35/00; C12Y 207/07001; C12N 2310/14; C12N 2310/531; C12N 2320/31

(57) ABSTRACT

The invention relates to methods and pharmaceutical compositions for the treatment of cancer, particularly BRCA-associated cancer and chemo-resistance BRCA-associated cancer. The inventors investigated the role of NMNAT1 in cancer, particularly in BRCA-associated cancer. The inventors demonstrated that NMNAT1 inhibition kills BRCA1 and BRCA2-mutated tumor cells but does not affect the survival of non-BRCA-mutated cells. The inventors also demonstrated that NMNAT1, a nuclear enzyme other than PARP1, is crucial for the survival of HRD cells and indicate that NMNAT1 is a key factor which activities are necessary for the survival of HRD cells. The inventors also demonstrated that inhibition of NMNAT1 kills PARP-inhibitor and cisplatin-resistant BRCA1 and BRCA2-mutated tumors and show that targeting NMNAT1 kills chemo-resistance HRD cells, particularly PARPi-resistant HRD cells. Altogether, the present invention highlights the role of NMNAT1 inhibitors in cancer and the use of NMNAT1 inhibitors in the treatment of cancer, particularly BRCA-associated cancer including BRCA-associated cancer with acquired drug resistance in mono- or combination therapy with PARPi. In the present invention, the inventors provide in vitro evidences towards a direct role of NMNAT1 in BRCA-associated cancer. Thus, the present invention relates to NMNAT1 inhibitor for use in the treatment of cancer, particularly HRD cancer. BRCA-associated cancer and chemo-resistance BRCA-associated cancer.

### FIELD OF THE INVENTION

The invention relates to methods and pharmaceutical compositions for the treatment of cancer, particularly HRD cancer, BRCA-associated cancer and chemo-resistance BRCA-associated cancer.

### BACKGROUND OF THE INVENTION

In the last decades, many efforts have been undertaken to gain mechanistic insights into the synthetic lethality between PARP1 and BRCA1/2 genes (Maya-Mendoza et al., 2018; Hanzlikova et al., 2018). Deficiencies in homologous recombination (HR)-mediated DNA repair occur mainly through genetic inactivation of BRCA1 and BRCA2 (BRCA1/2) genes and play a role in the initiation and progression of many tumor types. Defective HR (HRD) causes genomic instability and hyper-dependence on alternative DNA repair mechanisms for survival, setting the stage for synthetic lethality-based targeted therapy, as exemplified by the extreme sensitivity of HRD tumors to poly(ADP-ribose) polymerase inhibitors (PARPi) (Bryant et al., 2005; Farmer et al., 2005). This lethal interaction is successfully exploited in the clinic since the approval of PARP inhibitors (PARPi) for the treatment of BRCA1/2-mutated tumors (Patel et al., 2021), also referred as homologous recombination (HR)-deficient tumors (HRD). Various trials have been devised to evaluate PARPi effectiveness for patients with breast, ovarian, and prostate tumors harboring BRCA1/2 mutations (Pujade-Lauraine et al., 2017). However, PARPi and other chemotherapeutics have shown limited effectiveness in achieving HRD cancer remission, notably because drug resistance emerges and resistance to chemotherapy is emerging as the major obstacle to clinic effectiveness (Gogola et al., 2019). Hence, no therapeutic options are left for these patients, stressing the need for alternative therapeutic options (Konstantinopoulos et al., 2015).

The inventors discovered a new synthetic lethal interaction between NMNAT1 and BRCA1/2 proteins and show that the enzyme NMNAT1 (for nicotinamide mononucleotide adenylyltransferase 1), key to nuclear NAD biogenesis, maintains genomic stability in BRCA1/2-mutated cells. Nicotinamide (NAM) adenine dinucleotide (NAD+) biosynthesis occurs through multiple pathways, including de novo synthesis and salvage pathways (Cambronne et al., 2020). The NMNAT family includes enzymes essential for both these pathways and consists of three members (NMNAT1-3) with distinct subcellular distribution patterns in humans (Lau et al., 2009). NMNAT1 localizes to the nucleus, whereas the other two isozymes are found in the Golgi and the cytoplasm (NMNAT2) and the mitochondria (NMNAT3). All three members catalyze the same adenosine triphosphate (ATP)-dependent adenylation of NAM mononucleotide (NMN) or NAM acid mononucleotide (NaMN). NAD+ is a cofactor in cellular redox reactions of various energy producing metabolic pathways, such as glycolysis, tricarboxylic acide (TCA) cycle and oxidative phosphorylation. Here, NAD+ is reduced to NADH without being catabolized. However, in other subcellular compartments, NAD+ is consumed. In the nucleus, NAD+ is used as a substrate for protein modifications such as poly(ADP-ribosyl)ation (PAR) by PARP enzymes (PARP1 or PARP2), and protein deacetylation/mono(ADP-ribosyl) ation, thus having an impact on signaling, transcription and DNA repair. For example, PARP enzymes hydrolyze NAD+ to release ADPR groups that are used for the covalent mono- or poly(ADP-ribosyl) ation of proteins, DNA and RNA (Gibson et al., 2012; Gupte et al., 2017). Finally NAD+ can be consumed by the glycohydrolases CD38 and SARM1 (Cambronne et al., 2020).

Here, to find alternative curative options for BRCA1/2-mutated tumors, and the ones that have acquired PARPi resistance, the inventors investigated the role of NMNAT1 in cancer, particularly in BRCA-associated cancer and PARPi resistance. The inventors show that NMNAT1, the enzyme synthesizing nuclear NAD+ is synthetically lethal with BRCA1/2 through its function in base-excision repair. Consequently, inhibition of NMNAT1 not only kills HRD cells, but also the PARPi- and platinum-resistant ones, regardless the mechanism of resistance.

There is no disclosure in the art of the role of NMNAT1 inhibitor in cancer, particularly Homologous recombination deficiency (HRD) cancer and BRCA-associated cancer, and the targeting of NMNAT1 in the treatment of cancer, particularly HRD cancer and BRCA-associated cancer.

### SUMMARY OF THE INVENTION

The invention relates to methods and pharmaceutical compositions for the treatment of cancer, particularly HRD cancer and BRCA-associated cancer. In particular, the invention is defined by the claims.

### DETAILED DESCRIPTION OF THE INVENTION

The inventors investigated the role of NMNAT1 in cancer, particularly in BRCA-associated cancer. The inventors demonstrated that NMNAT1 inhibition kills BRCA1 and BRCA2-mutated tumor cells but does not affect the survival of non-BRCA-mutated cells. The inventors also demonstrated that NMNAT1, a nuclear enzyme other than PARP1, is crucial for the survival of HRD cells and indicate that NMNAT1 is a key factor which activities are necessary for the survival of HRD cells. The inventors also demonstrated that inhibition of NMNAT1 kills PARP-inhibitor and cisplatin-resistant BRCA1 and BRCA2-mutated tumors and show that targeting NMNAT1 kills chemo-resistance HRD cells, particularly PARPi-resistant HRD cells. Altogether, the present invention highlights the role of NMNAT1 inhibitors in cancer and the use of NMNAT1 inhibitors in the treatment of cancer, particularly BRCA-associated cancer including BRCA-associated cancer with acquired drug resistance in mono- or combination therapy with PARPi.

Accordingly, the invention relates to the targeting of NMNAT1 in the treatment of cancer, particularly HRD cancer, BRCA-associated cancer and chemo-resistance BRCA-associated cancer.

### Therapeutic Method

Accordingly, in a first aspect, the invention relates to a NMNAT1 inhibitor for use in the treatment of cancer.

In some embodiment, the invention relates to NMNAT1 inhibitor for use in the treatment of HRD cancer.

In some embodiment, the invention relates to NMNAT1 inhibitor for use in the treatment of BRCA-associated cancer.

In some embodiment, the invention relates to NMNAT1 inhibitor for use in the treatment of BRCA-associated cancer, particularly chemo-resistance BRCA-associated cancer such as PARPi resistant BRCA-associated cancer or cisplatin resistant BRCA-associated cancer.

As used herein, the terms “subject”, “individual” or “patient” are interchangeable and refer to a mammal. Typically, a subject according to the invention refers to any subject, preferably human. In a particular embodiment, the term “subject” refers to a subject afflicted or at risk to be afflicted with cancer. In a particular embodiment, the term “subject” refers to a subject afflicted or at risk to be afflicted with HRD cancer. In a particular embodiment, the term “subject” refers to a subject afflicted or at risk to be afflicted with BRCA-associated cancer. In some embodiment, the term “subject” refers to a subject afflicted or at risk to be afflicted with chemo-resistance BRCA-associated cancer. In some embodiment, the term “subject” refers to a subject afflicted or at risk to be afflicted with BRCA-associated cancer such as Homologous recombination deficiency (HRD) and/or BRCA-deficiency (basal-like, luminal, and HER2-overexpressing breast carcinomas and other cancers) and breast, ovarian, prostate, pancreatic or any other type of tumors harboring BRCA1/2 mutations.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “cancer” refers to any cancer that may affect any one of the following tissues or organs: breast; liver; kidney; heart, mediastinum, pleura; floor of mouth; lip; salivary glands; tongue; gums; oral cavity; palate; tonsil; larynx; trachea; bronchus, lung; pharynx, hypopharynx, oropharynx, nasopharynx; esophagus; digestive organs such as stomach, intrahepatic bile ducts, biliary tract, pancreas, small intestine, colon; rectum; urinary organs such as bladder, gallbladder, ureter; rectosigmoid junction; anus, anal canal; skin; bone; joints, articular cartilage of limbs; eye and adnexa; brain; peripheral nerves, autonomic nervous system; spinal cord, cranial nerves, meninges; and various parts of the central nervous system; connective, subcutaneous and other soft tissues; retroperitoneum, peritoneum; adrenal gland; thyroid gland; endocrine glands and related structures; female genital organs such as ovary, uterus, cervix uteri; corpus uteri, vagina, vulva; male genital organs such as penis, testis and prostate gland; hematopoictic and reticuloendothelial systems; blood; lymph nodes; thymus.

The term “cancer” according to the invention comprises leukemias, seminomas, melanomas, teratomas, lymphomas, non-Hodgkin lymphoma, neuroblastomas, gliomas, adenocarcinoma, mesothelioma (including pleural mesothelioma, peritoneal mesothelioma, pericardial mesothelioma and end stage mesothelioma), rectal cancer, endometrial cancer, thyroid cancer (including papillary thyroid carcinoma, follicular thyroid carcinoma, medullary thyroid carcinoma, undifferentiated thyroid cancer, multiple endocrine neoplasia type 2A, multiple endocrine neoplasia type 2B, familial medullary thyroid cancer, pheochromocytoma and paraganglioma), skin cancer (including malignant melanoma, basal cell carcinoma, squamous cell carcinoma, Karposi's sarcoma, keratoacanthoma, moles, dysplastic nevi, lipoma, angioma and dermatofibroma), nervous system cancer, brain cancer (including astrocytoma, medulloblastoma, glioma, lower grade glioma, ependymoma, germinoma (pincaloma), glioblastoma multiform, oligodendroglioma, schwannoma, retinoblastoma, congenital tumors, spinal cord neurofibroma, glioma or sarcoma), skull cancer (including osteoma, hemangioma, granuloma, xanthoma or osteitis deformans), meninges cancer (including meningioma, meningiosarcoma or gliomatosis), head and neck cancer (including head and neck squamous cell carcinoma and oral cancer (such as, e.g., buccal cavity cancer, lip cancer, tongue cancer, mouth cancer or pharynx cancer)), lymph node cancer, gastrointestinal cancer, liver cancer (including hepatoma, hepatocellular carcinoma, cholangiocarcinoma, hepatoblastoma, angiosarcoma, hepatocellular adenoma and hemangioma), colon cancer, stomach or gastric cancer, esophageal cancer (including squamous cell carcinoma, larynx, adenocarcinoma, leiomyosarcoma or lymphoma), colorectal cancer, intestinal cancer, small bowel or small intestines cancer (such as, e.g., adenocarcinoma lymphoma, carcinoid tumors, Karposi's sarcoma, leiomyoma, hemangioma, lipoma, neurofibroma or fibroma), large bowel or large intestines cancer (such as, e.g., adenocarcinoma, tubular adenoma, villous adenoma, hamartoma or leiomyoma), pancreatic cancer (including ductal adenocarcinoma, insulinoma, glucagonoma, gastrinoma, carcinoid tumors or vipoma), car, nose and throat (ENT) cancer, breast cancer (including HER2-enriched breast cancer, luminal A breast cancer, luminal B breast cancer and triple negative breast cancer), cancer of the uterus (including endometrial cancer such as endometrial carcinomas, endometrial stromal sarcomas and malignant mixed Müllerian tumors, uterine sarcomas, leiomyosarcomas and gestational trophoblastic disease), ovarian cancer (including dysgerminoma, granulosa-theca cell tumors and Sertoli-Leydig cell tumors), cervical cancer, vaginal cancer (including squamous-cell vaginal carcinoma, vaginal adenocarcinoma, clear cell vaginal adenocarcinoma, vaginal germ cell tumors, vaginal sarcoma botryoides and vaginal melanoma), vulvar cancer (including squamous cell vulvar carcinoma, verrucous vulvar carcinoma, vulvar melanoma, basal cell vulvar carcinoma, Bartholin gland carcinoma, vulvar adenocarcinoma and erythroplasia of Queyrat), genitourinary tract cancer, kidney cancer (including clear renal cell carcinoma, chromophobe renal cell carcinoma, papillary renal cell carcinoma, adenocarcinoma, Wilm's tumor, nephroblastoma, lymphoma or leukemia), adrenal cancer, bladder cancer, urethra cancer (such as, e.g., squamous cell carcinoma, transitional cell carcinoma or adenocarcinoma), prostate cancer (such as, e.g., adenocarcinoma or sarcoma) and testis cancer (such as, e.g., seminoma, teratoma, embryonal carcinoma, teratocarcinoma, choriocarcinoma, sarcoma, interstitial cell carcinoma, fibroma, fibroadenoma, adenomatoid tumors or lipoma), lung cancer (including small cell lung carcinoma (SCLC), non-small cell lung carcinoma (NSCLC) including squamous cell lung carcinoma, lung adenocarcinoma (LUAD), and large cell lung carcinoma, bronchogenic carcinoma, alveolar carcinoma, bronchiolar carcinoma, bronchial adenoma, lung sarcoma, chondromatous hamartoma and pleural mesothelioma), sarcomas (including Askin's tumor, sarcoma botryoides, chondrosarcoma, Ewing's sarcoma, malignant hemangioendothelioma, malignant schwannoma, osteosarcoma and soft tissue sarcomas), soft tissue sarcomas (including alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, plexiform fibrohistiocytic tumor, rhabdomyosarcoma, synovial sarcoma and undifferentiated pleomorphic sarcoma, cardiac cancer (including sarcoma such as, e.g., angiosarcoma, fibrosarcoma, rhabdomyosarcoma or liposarcoma, myxoma, rhabdomyoma, fibroma, lipoma and teratoma), bone cancer (including osteogenic sarcoma, osteosarcoma, fibrosarcoma, malignant fibrous histiocytoma, chondrosarcoma, Ewing's sarcoma, malignant lymphoma and reticulum cell sarcoma, multiple myeloma, malignant giant cell tumor chordoma, osteochronfroma, ostcocartilaginous exostoses, benign chondroma, chondroblastoma, chondromyxoid fibroma, osteoid osteoma and giant cell tumors), hematologic and lymphoid cancer, blood cancer (including acute myeloid leukemia, chronic myeloid leukemia, acute lymphoblastic leukemia, chronic lymphocytic leukemia, myeloproliferative diseases, multiple myeloma and myelodysplasia syndrome), Hodgkin's disease, non-Hodgkin's lymphoma and hairy cell and lymphoid disorders, and the metastases thereof.

The term “homologous recombination deficiency cancer” or “HRD cancer” has its general meaning in the art and refers to cancer displaying defective homologous recombination (HRD)-mediated DNA repair which causes genomic instability and hyper-dependence on alternative DNA repair mechanisms for survival. The term “HRD cancer” include but is not limited to BRCA-associated cancer.

The term “BRCA-associated cancer” has its general meaning in the art and refers to cancer associated with BRCA mutation or BRCA expression deficiency. The term “BRCA-associated cancer” refers to cancer selected from but not limited to cancer associated with BRCA1 and/or BRCA2 mutation, in particular inactivation of BRCA1 and/or BRCA2 genes, cancer associated with BRCA1 and/or BRCA2 expression deficiency, homologous recombination deficiency (HRD) cancer and/or BRCA-deficiency cancer (such as basal-like, luminal, and HER2-overexpressing carcinomas, breast, ovarian, and prostate tumors harboring BRCA1/2 mutations and other cancers). In some embodiment, the term “BRCA-associated cancer” refers to breast cancer, ovary cancer, cervix cancer, pancreas cancer, lung cancer, head and neck cancer and melanoma with BRCA1 and/or BRCA2 mutation or BRCA1 and/or BRCA2 expression deficiency.

As used herein “BRCA1/2” denotes BRCA1 and/or BRCA2, more particularly BRCA1 and BRCA2.

The term “BRCA-associated cancer” also refers to chemo-resistance BRCA-associated cancer such as PARP inhibitor (PARPi) resistant BRCA-associated cancer, PARPi-resistant HRD tumors including tumors with somatic reversion of BRCA1/2 mutation and subsequent HR restoration, cisplatin resistant BRCA-associated cancer and cisplatin-resistant BRCA1 and BRCA2-mutated tumors including tumors with somatic reversion of BRCA1/2 mutation and subsequent HR restoration.

The term “PARP inhibitor” or “PARPi” has its general meaning in the art and refers to PARP inhibitor such as olaparib, rucaparib, niraparib and talazoparib. The term “PARP inhibitor” also refers to PARP inhibitor such iniparib, veliparib, Pamiparib (BGB-290), CEP 9722, E7016 and 3-Aminobenzamide.

The term “NMNAT1” has its general meaning in the art and refers to a Nicotinamide Nucleotide Adenylyltransferase 1, also known as Nicotinamide/nicotinic acid mononucleotide adenylyltransferase 1 (Protein Accession number Q9HAN9).

As used herein, the term “NMNAT1 inhibitor” refers to any compound selected from the group consisting of but not limited to compounds targeting Nicotinamide Nucleotide Adenylyltransferase 1. The term “NMNAT1 inhibitor” refers to compounds that bind to NMNAT1 and function as potent antagonists of NMNAT1. The term “NMNAT1 inhibitor” has its general meaning in the art and refers to a compound that selectively inactivates NMNAT1. Typically, a NMNAT1 inhibitor is a small organic molecule, a polypeptide, an aptamer, an oligonucleotide (antisense oligonucleotides, siRNA, shRNA, DNA and RNA aptamers), or an antibody. NMNAT1 inhibitors are well-known in the art as such as described in WO2013/130672; Kusumanchi et al., 2013.

The term “NMNAT1 inhibitor” refers to any compound selected from but not limited to siRNA such s34981 (hNMNAT1 siRNA) (Kusumanchi et al., 2013; WO2013/130672), siRNA ID130437, siRNA ID130438, siRNA ID130439, siRNA ID212976, siRNA ID212977 (Thermo Fisher Scientific).

In some embodiment, the “NMNAT1 inhibitor” refers to siRNA targeting the NMNAT1 sequences SEQ ID NO: 1 (The target sequences of the used siRNA NMNAT1 #2 (Qiagen)) and SEQ ID NO: 2 (The target sequences of the used siRNA NMNAT1 #8 (Dharmacon)) and shRNA targeting the NMNAT1 sequences SEQ ID NO: 3 (The target sequences of the used pLKO.1 plasmid carrying the shRNA NMNAT1 (Sigma)) and the pTRIPZ plasmid carrying the doxycycline-inducible shRNA targeting human NMNAT1 (clone id: V3THS_315166) (Horizon).

SEQ ID NO: 1
GGAAGGAAGAGGAAGTGGACT
SEQ ID NO: 2
ACAGGAAGGTACACAGTTG
SEQ ID NO: 3
CGCTACTTGGTACCAGATCTT

Tests and assays for determining whether a compound is a NMNAT1 inhibitor are well known by the skilled person in the art such as described in WO2013/130672; Kusumanchi et al., 2013. Determining whether a compound is a NMNAT1 inhibitor may also be performed using by using recombinant NMNAT1 proteins, measuring NAD⁺ production in vitro, competitive binding assays and measuring the binding affinities, measuring nuclear NAD⁺ concentrations, nuclear PARylation levels, measuring synthetic lethal interaction between NMNAT1 and BRCA1/2 proteins or assays such as described in the examples.

In another embodiment, the compound of the invention is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against the target of the invention as above described, the skilled man in the art can easily select those blocking or inactivating the target.

In another embodiment, the compound of the invention is an antibody (the term including “antibody portion”) directed against the target.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of the target. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the antigen may be provided as synthetic peptides corresponding to antigenic regions of interest in the target. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FRI through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria, which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. Sec, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′)2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4. In a preferred embodiment, the compound of the invention is a Human IgG4.

In another embodiment, the antibody according to the invention is a single domain antibody. The term “single domain antibody” (sdAb) or “VHH” refers to the single heavy chain variable domain of antibodies of the type that can be found in Camelid mammals, which are naturally devoid of light chains. Such VHH are also called “Nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In another aspect, the invention provides an antibody that competes for binding to the target with the antibody of the invention.

As used herein, the term “binding” in the context of the binding of an antibody to a predetermined antigen or epitope typically is a binding with an affinity corresponding to a KD of about 10-7 M or less, such as about 10-8 M or less, such as about 10-9 M or less, about 10-10 M or less, or about 10-11 M or even less when determined by for instance surface plasmon resonance (SPR) technology in a BIAcore 3000 instrument using a soluble form of the antigen as the ligand and the antibody as the analyte. BIACORE® (GE Healthcare, Piscaataway, NJ) is one of a variety of surface plasmon resonance assay formats that are routinely used to epitope bin panels of monoclonal antibodies. Typically, an antibody binds to the predetermined antigen with an affinity corresponding to a KD that is at least ten-fold lower, such as at least 100-fold lower, for instance at least 1,000-fold lower, such as at least 10,000-fold lower, for instance at least 100,000-fold lower than its KD for binding to a non-specific antigen (e.g., BSA, casein), which is not identical or closely related to the predetermined antigen. When the KD of the antibody is very low (that is, the antibody has a high affinity), then the KD with which it binds the antigen is typically at least 10,000-fold lower than its KD for a non-specific antigen. An antibody is said to essentially not bind an antigen or epitope if such binding is either not detectable (using, for example, plasmon resonance (SPR) technology in a BIAcore 3000 instrument using a soluble form of the antigen as the ligand and the antibody as the analyte), or is 100 fold, 500 fold, 1000 fold or more than 1000 fold less than the binding detected by that antibody and an antigen or epitope having a different chemical structure or amino acid sequence.

Additional antibodies can be identified based on their ability to cross-compete (e.g., to competitively inhibit the binding of, in a statistically significant manner) with other antibodies of the invention in standard antigen binding assays. The ability of a test antibody to inhibit the binding of antibodies of the present invention to the target demonstrates that the test antibody can compete with that antibody for binding to the target; such an antibody may, according to non-limiting theory, bind to the same or a related (e.g., a structurally similar or spatially proximal) epitope on the target as the antibody with which it competes. Thus, another aspect of the invention provides antibodies that bind to the same antigen as, and compete with, the antibodies disclosed herein. As used herein, an antibody “competes” for binding when the competing antibody inhibits the target binding of an antibody or antigen binding fragment of the invention by more than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% in the presence of an equimolar concentration of competing antibody.

In other embodiments the antibodies or antigen binding fragments of the invention bind to one or more epitopes of the target. In some embodiments, the epitopes to which the present antibodies or antigen binding fragments bind are linear epitopes. In other embodiments, the epitopes to which the present antibodies or antigen binding fragments bind are non-linear, conformational epitopes.

In one embodiment, the NMNAT1 inhibitor of the invention is a NMNAT1 expression inhibitor.

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include messenger RNAs, which are modified, by processes such as capping, polyadenylation, methylation, and editing, and proteins modified by, for example, methylation, acetylation, phosphorylation, ubiquitination, SUMOylation, ADP-ribosylation, myristilation, and glycosylation.

An “inhibitor of expression” refers to a natural or synthetic compound that has a biological effect to inhibit the expression of a gene. An “inhibitor of expression” refers to any compound that has a biological effect to inhibit the expression of a target gene and/or the expression of target protein. In one embodiment of the invention, said inhibitor of expression is a short hairpin RNA (shRNA), a small inhibitory RNA (siRNA), or an antisense oligonucleotide. Preferably, the inhibitor of expression is a siRNA or a shRNA.

The target expression inhibitors for use in the present invention may be based on antisense oligonucleotide constructs. Anti-sense oligonucleotides, including anti-sense RNA molecules and anti-sense DNA molecules, would act to directly block the translation of the target mRNA by binding thereto and thus preventing protein translation or increasing mRNA degradation, thus decreasing the level of the target proteins, and thus activity, in a cell. For example, antisense oligonucleotides of at least about 15 bases and complementary to unique regions of the mRNA transcript sequence encoding the target can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as a target expression inhibitors for use in the present invention. The target gene expression can be reduced by contacting the subject or cell with a small double stranded RNA (dsRNA), or a vector or construct causing the production of a small double stranded RNA, such that the target expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, MT. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Short hairpin RNA (shRNA) or Small inhibitory RNAs (siRNAs) can function as inhibitors of gene expression for use in the invention. Gene expression can be reduced with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known.

Ribozymes can also function as target expression inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of the target mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides (ODNs) and ribozymes useful as target inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing the target. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a further aspect, the invention relates to a method of treating cancer in a subject in need thereof, comprising administering to the subject a therapeutically effective amount of a NMNAT1 inhibitor.

In some embodiments, the invention relates to a method of treating HRD cancer in a subject in need thereof, comprising administering to the subject a therapeutically effective amount of a NMNAT1 inhibitor.

In some embodiments, the invention relates to a method of treating BRCA-associated cancer in a subject in need thereof, comprising administering to the subject a therapeutically effective amount of a NMNAT1 inhibitor.

In some embodiments, the invention relates to a method of treating BRCA-associated cancer, particularly chemo-resistance BRCA-associated cancer such as PARPi resistant BRCA-associated cancer or cisplatin resistant BRCA-associated cancer in a subject in need thereof, comprising administering to the subject a therapeutically effective amount of a NMNAT1 inhibitor.

In some embodiments, the NMNAT1 inhibitor and/or pharmaceutical composition according to the invention is administered in combination with PARPi such as olaparib, rucaparib, niraparib, talazoparib, iniparib, veliparib, Pamiparib (BGB-290), CEP 9722, E7016, E7449 and 3-Aminobenzamide.

In some embodiments, the NMNAT1 inhibitor and/or pharmaceutical composition according to the invention is administered in combination with cisplatin.

In some embodiments, the NMNAT1 inhibitor and/or pharmaceutical composition according to the invention is administered in combination with Pole inhibitors such as novobiocin.

In some embodiments, the NMNAT1 inhibitor and/or pharmaceutical composition according to the invention is administered in combination with cancer therapies. In particular, compound and/or pharmaceutical composition of the invention may be administered in combination with targeted therapy, immunotherapy such as immune checkpoint therapy and immune checkpoint inhibitor, co-stimulatory antibodies, chemotherapy and/or radiotherapy.

As used herein, the term “immunotherapy” refers to a cancer therapeutic treatment using the immune system to reject cancer. The therapeutic treatment stimulates the patient's immune system to attack the malignant tumor cells.

Immune checkpoint therapy such as checkpoint inhibitors include, but are not limited to programmed death-1 (PD-1) inhibitors, programmed death ligand-1 (PD-L1) inhibitors, programmed death ligand-2 (PD-L2) inhibitors, lymphocyte-activation gene 3 (LAG3) inhibitors, T-cell immunoglobulin and mucin-domain containing protein 3 (TIM-3) inhibitors, T cell immunoreceptor with Ig and ITIM domains (TIGIT) inhibitors, B- and T-lymphocyte attenuator (BTLA) inhibitors, V-domain Ig suppressor of T-cell activation (VISTA) inhibitors, cytotoxic T-lymphocyte-associated protein 4 (CTLA4) inhibitors, Indolcamine 2,3-dioxygenase (IDO) inhibitors, killer immunoglobulin-like receptors (KIR) inhibitors, KIR2L3 inhibitors, KIR3DL2 inhibitors and carcinoembryonic antigen-related cell adhesion molecule 1 (CEACAM-1) inhibitors. In particular, checkpoint inhibitors include antibodies anti-PD1, anti-PD-L1, anti-CTLA-4, anti-TIM-3, anti-LAG3. Immune checkpoint therapy also include co-stimulatory antibodies delivering positive signals through immune-regulatory receptors including but not limited to ICOS, CD137, CD27, OX-40 and GITR.

Example of anti-PD1 antibodies include, but are not limited to, nivolumab, cemiplimab (REGN2810 or REGN-2810), tislelizumab (BGB-A317), tislelizumab, spartalizumab (PDR001 or PDR-001), ABBV-181, JNJ-63723283, BI 754091, MAG012, TSR-042, AGEN2034, pidilizumab, nivolumab (ONO-4538, BMS-936558, MDX1106, GTPL7335 or Opdivo), pembrolizumab (MK-3475, MK03475, lambrolizumab, SCH-900475 or Keytruda) and antibodies described in International patent applications WO2004004771, WO2004056875, WO2006121168, WO2008156712, WO2009014708, WO2009114335, WO2013043569 and WO2014047350. Example of anti-PD-L1 antibodies include, but are not limited to, LY3300054, atczolizumab, durvalumab and avelumab. Example of anti-CTLA-4 antibodies include, but are not limited to, ipilimumab (see, e.g., US patents U.S. Pat. Nos. 6,984,720 and 8,017,114), tremelimumab (see, e.g., US patents U.S. Pat. Nos. 7,109,003 and 8,143,379), single chain anti-CTLA4 antibodies (see, e.g., International patent applications WO1997020574 and WO2007123737) and antibodies described in US patent U.S. Pat. No. 8,491,895. Example of anti-VISTA antibodies are described in US patent application US20130177557. Example of inhibitors of the LAG3 receptor are described in US patent U.S. Pat. No. 5,773,578. Example of KIR inhibitor is IPH4102 targeting KIR3DL2.

In some embodiments, the compound and/or pharmaceutical composition of the invention may be used in combination with targeted therapy. As used herein, the term “targeted therapy” refers to targeted therapy agents, drugs designed to interfere with specific molecules necessary for tumor growth and progression. For example, targeted therapy agents such as therapeutic monoclonal antibodies target specific antigens found on the cell surface, such as transmembrane receptors or extracellular growth factors. Small molecules can penetrate the cell membrane to interact with targets inside a cell. Small molecules are usually designed to interfere with the enzymatic activity of the target protein such as for example proteasome inhibitor, tyrosine kinase or cyclin-dependent kinase inhibitor, histone deacetylase inhibitor. Targeted therapy may also use cytokines. Examples of such targeted therapy include with no limitations: Ado-trastuzumab emtansine (HER2), Afatinib (EGFR (HER1/ERBB1), HER2), Aldesleukin (Proleukin), alectinib (ALK), Alemtuzumab (CD52), axitinib (kit, PDGFRbeta, VEGFR1/2/3), Belimumab (BAFF), Belinostat (HDAC), Bevacizumab (VEGF ligand), Blinatumomab (CD19/CD3), bortezomib (proteasome), Brentuximab vedotin (CD30), bosutinib (ABL), brigatinib (ALK), cabozantinib (FLT3, KIT, MET, RET, VEGFR2), Canakinumab (IL-1 beta), carfilzomib (proteasome), ceritinib (ALK), Cetuximab (EGFR), cofimctinib (MEK), Crizotinib (ALK, MET, ROS1), Dabrafenib (BRAF), Daratumumab (CD38), Dasatinib (ABL), Denosumab (RANKL), Dinutuximab (B4GALNT1 (GD2)), Elotuzumab (SLAMF7), Enasidenib (IDH2), Erlotinib (EGFR), Everolimus (mTOR), Gefitinib (EGFR), Ibritumomab tiuxetan (CD20), Sonidegib (Smoothened), Sipuleucel-T, Siltuximab (IL-6), Sorafenib (VEGFR, PDGFR, KIT, RAF), (Tocilizumab (IL-6R), Temsirolimus (mTOR), Tofacitinib (JAK3), Tramctinib (MEK), Tositumomab (CD20), Trastuzumab (HER2), Vandetanib (EGFR), Vemurafenib (BRAF), Venetoclax (BCL2), Vismodegib (PTCH, Smoothened), Vorinostat (HDAC), Ziv-aflibercept (PIGF, VEGFA/B), Olaparib (PARP inhibitor).

In some embodiments, the compound and/or pharmaceutical composition of the invention may be used in combination with chemotherapy. As used herein, the term “antitumor chemotherapy” or “chemotherapy” has its general meaning in the art and refers to a cancer therapeutic treatment using chemical or biochemical substances, in particular using one or several antineoplastic agents or chemotherapeutic agents. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trictylenephosphoramide, tricthiylencthiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin;

nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynamicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitracrine; pentostatin; phenamet; pirarubicin; losoxantrone; podophyllinic acid; 2-ethylhydrazide; methylhydrazine derivatives including N-methylhydrazine (MIH) and procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; anthracyclines, nitrosoureas, antimetabolites, epipodophylotoxins, enzymes such as L-asparaginase; anthracenediones; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide; and pharmaceutically acceptable salts, acids or derivatives of any of the above.

In some embodiments, the compound and/or pharmaceutical composition of the invention is administered to the subject in combination with radiotherapy. Suitable examples of radiation therapies include, but are not limited to external beam radiotherapy (such as superficial X-rays therapy, orthovoltage X-rays therapy, megavoltage X-rays therapy, radiosurgery, stereotactic radiation therapy, Fractionated stereotactic radiation therapy, cobalt therapy, electron therapy, fast neutron therapy, neutron-capture therapy, proton therapy, intensity modulated radiation therapy (IMRT), 3-dimensional conformal radiation therapy (3D-CRT) and the like); brachytherapy; unscaled source radiotherapy; tomotherapy; and the like. Gamma rays are another form of photons used in radiotherapy. Gamma rays are produced spontaneously as certain elements (such as radium, uranium, and cobalt 60) release radiation as they decompose, or decay. In some embodiments, radiotherapy may be proton radiotherapy or proton minibeam radiation therapy. Proton radiotherapy is an ultra-precise form of radiotherapy that uses proton beams (Prezado Y, Jouvion G, Guardiola C, Gonzalez W, Juchaux M, Bergs J, Nauraye C, Labiod D, De Marzi L, Pouzoulet F, Patriarca A, Dendale R. Tumor Control in RG2 Glioma-Bearing Rats: A Comparison Between Proton Minibeam Therapy and Standard Proton Therapy. Int J Radiat Oncol Biol Phys. 2019 Jun. 1; 104 (2): 266-271. doi: 10.1016/j.ijrobp.2019.01.080; Prezado Y, Jouvion G, Patriarca A, Nauraye C, Guardiola C, Juchaux M, Lamirault C, Labiod D, Jourdain L, Sebrie C, Dendale R, Gonzalez W, Pouzoulet F. Proton minibeam radiation therapy widens the therapeutic index for high-grade gliomas. Sci Rep. 2018 Nov. 7; 8 (1): 16479. doi: 10.1038/s41598-018-34796-8). Radiotherapy may also be FLASH radiotherapy (FLASH-RT) or FLASH proton irradiation. FLASH radiotherapy involves the ultra-fast delivery of radiation treatment at dose rates several orders of magnitude greater than those currently in routine clinical practice (ultra-high dose rate) (Favaudon V, Fouillade C, Vozenin M C. The radiotherapy FLASH to save healthy tissues. Med Sci (Paris) 2015; 31:121-123. DOI: 10.1051/medsci/20153102002); Patriarca A., Fouillade C. M., Martin F., Pouzoulet F., Nauraye C., et al. Experimental set-up for FLASH proton irradiation of small animals using a clinical system. Int J Radiat Oncol Biol Phys, 102 (2018), pp. 619-626. doi: 10.1016/j.ijrobp.2018.06.403. Epub 2018 Jul. 11).

### Pharmaceutical Composition

The compounds of the invention may be used or prepared in a pharmaceutical composition.

In one embodiment, the invention relates to a pharmaceutical composition comprising the compound of the invention and a pharmaceutical acceptable carrier for use in the treatment of cancer in a subject of need thereof.

In some embodiments, the invention relates to a pharmaceutical composition comprising the compound of the invention and a pharmaceutical acceptable carrier for use in the treatment of HRD cancer in a subject in need thereof.

In some embodiments, the invention relates to a pharmaceutical composition comprising the compound of the invention and a pharmaceutical acceptable carrier for use in the treatment of BRCA-associated cancer in a subject in need thereof.

In some embodiment, the invention relates to a pharmaceutical composition comprising the compound of the invention and a pharmaceutical acceptable carrier for use in the treatment of BRCA-associated cancer, particularly chemo-resistance BRCA-associated cancer such as PARPi resistant BRCA-associated cancer or cisplatin resistant BRCA-associated cancer.

Typically, the compound of the invention may be combined with pharmaceutically acceptable excipients, and optionally sustained-release matrices, such as biodegradable polymers, to form therapeutic compositions.

Typically, the compounds according to the invention as described above are administered to the subject in a therapeutically effective amount.

By a “therapeutically effective amount” of the compound of the present invention as above described is meant a sufficient amount of the compound at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the compound of the present invention for the symptomatic adjustment of the dosage to the patient to be treated. A medicament typically contains from about 0.01 mg to about 500 mg of the compound of the present invention, preferably from 1 mg to about 100 mg of the compound of the present invention. An effective amount of the drug is ordinarily supplied at a dosage level from 0.0002 mg/kg to about 20 mg/kg of body weight per day, especially from about 0.001 mg/kg to 7 mg/kg of body weight per day.

In a particular embodiment, the compound according to the invention may be used in a concentration between 0.01 μM and 20 μM, particularly, the compound of the invention may be used in a concentration of 0.01, 0.05, 0.1, 0.5, 1.0, 2.5, 5.0, 10.0, 15.0, 20.0 μM.

According to the invention, the compound of the present invention is administered to the subject in the form of a pharmaceutical composition. Typically, the compound of the present invention may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The compound of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized agents of the present inventions into a sterile vehicle which contains the basic dispersion medium and the required other ingredients from those enumerated above. In the case of sterile powders for the preparation of sterile injectable solutions, the typical methods of preparation arc vacuum-drying and freeze-drying techniques which yield a powder of the compound of the present invention plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

Pharmaceutical compositions of the invention may include any further compound which is used in the treatment of cancer such as described above.

In some embodiments, the pharmaceutical compositions of the invention may include any further compound which is used in the treatment of BRCA-associated cancer.

In one embodiment, said additional active compounds may be contained in the same composition or administrated separately.

In another embodiment, the pharmaceutical composition of the invention relates to combined preparation for simultaneous, separate or sequential use in the treatment of cancer in a subject in need thereof.

In some embodiments, the pharmaceutical composition of the invention relates to combined preparation for simultaneous, separate or sequential use in the treatment of BRCA-associated cancer in a subject in need thereof.

The invention also provides kits comprising the compound of the invention. Kits containing the compound of the invention find use in therapeutic methods.

The invention will be further illustrated by the following figures and examples. However, these examples and figures should not be interpreted in any way as limiting the scope of the present invention.

### FIGURES

FIG. 1: a, Quantification (left) and representative images (right) of clonogenic formation of wild type (WT) and BRCA2^{-/-} (clones C1 and C2) RPE-1 cells with and without BRCA2 cDNA complementation, after transfection with the indicated NMNAT1 siRNAs. b, Clonogenic formation (left) and WB-analysis (right) of BRCA2 mAID HeLa cells in the presence or absence of auxin (IAA) after transduction with viral particles carrying NMNAT1 shRNA or control. c, Clonogenic formation of WT and BRCA1^{-/-} RPE-1 cells after transfection with siNMNAT1 #2. d, Clonogenic formation of WT and NMNAT1^{-/-} RPE-1 cells after transfection with the indicated BRCA1/2 siRNAs. e, Quantification (left) and representative images (right) of clonogenic formation of the indicated HR-proficient and HR-deficient cells upon siNMNAT1. f, DNA damage quantification by alkaline COMET assay of P53−/− RPE-1 cells in the presence or absence of NMNAT1. g, Survival assay of WT and NMNAT1^{-/-} RPE-1 cells exposed to the indicated doses of the PARP inhibitor rucaparib (PARPi).

FIG. 2: a,b Proliferation curve of WT and NMNAT1^{-/-} cells in P53^{-/-} (a) and P53++ (b) RPE-1. c, DNA damage quantification by alkaline COMET assay of the same cells as in a.b.

FIG. 3: a, WB analysis showing PARP1 activation in WT and NMNAT1^{-/-} RPE-1 cells (G9) complemented with wild type (WT), catalytically dead (MUT, W169A) NMNAT1 cDNA or empty vector (EV) following exposure to PARG inhibitor and MMS. b, DNA damage quantification by alkaline COMET assay of WT and NMNAT1^{-/-} (clone G9) RPE-1 cells complemented or not (EV) with wild type (WT) or catalytically dead (MUT, W169A) NMNAT1 cDNA. c,d, Clonogenic formation of cells as in b in response to PARPi (c) or following transfection with cither BRCA2 or control (siCTRL) siRNAs (d left panel). Representative images are shown in d right panel.

FIG. 4: a, Schematic of the drug-resistant cell lines used in this work. b, Clonogenic formation of wild type (WT), BRCA2^{-/-} (clones C1 and C2) and BRCA2^{-/-}- derived PARPi-resistant RPE-1 cells after control or NMNAT1 (siNMNAT1 #2) siRNAs transfection. c, Clonogenic formation of parental and derived PARPi-resistant CAPAN-1 cells after transduction with shNMNAT1 lentiviral particles. Representative images are shown in the right panel. d, Clonogenic formation of PEO-1 and PEO-4 after transduction with shNMNAT1 lentiviral particles. Lentiviral particles carrying scramble shRNA (shSCR) were used as control for all the experiments shown in c,d.

FIG. 5: NMNAT1 inhibition kills HRD tumors in vivo. A, Growth (relative tumor volume, RTV) of indicated OVCAR8 xenografts in vivo. The OVCAR8 is a BRCA1 deficient (hypermethylated promoter) human ovarian cell line, xenotransplanted subcutaneously in athymic nude mice. Doxycycline was supplemented in the food (625 p.p.m.). B, Growth (relative tumor volume, RTV) of indicated KURAMOCHI xenografts in vivo. The KURAMOCHI is a BRCA2 deficient (homozygous deletion) human ovarian cell line, xenotransplanted intraperitonealy in NSG mice. Doxycycline was given IP to the mice (indicated by arrow), at the concentration of 10 mg/Kg.

FIG. 6: NMNAT1 inhibition kills PARPi-resistant HRD tumors in vivo. A. Growth (relative tumor volume, RTV) of parental and PARPi-resistant OVCAR8 xenografts in vivo upon PARPi (rucaparib) or vehicle treatment. B, Growth (relative tumor volume, RTV) of PARPi-resistant OVCAR8 xenografts in vivo. C, Relative tumour volumes (RTV) for individual mice treated in B after five weeks of treatment. D, Overall survival for mice bearing PARPi-resistant OVCAR8 xenografts treated with vehicle or doxycycline. For data shown in FIG. 6, the PARPi-resistant OVCAR8 cells were xenotransplanted subcutaneously in athymic nude mice. Doxycycline was supplemented in the food (625 p.p.m.).

### EXAMPLE

### Results

NMNAT1 Inhibition Kills BRCA1 and BRCA2-Mutated Tumor Cells but does not Affect the Survival of Non-BRCA-Mutated Cells

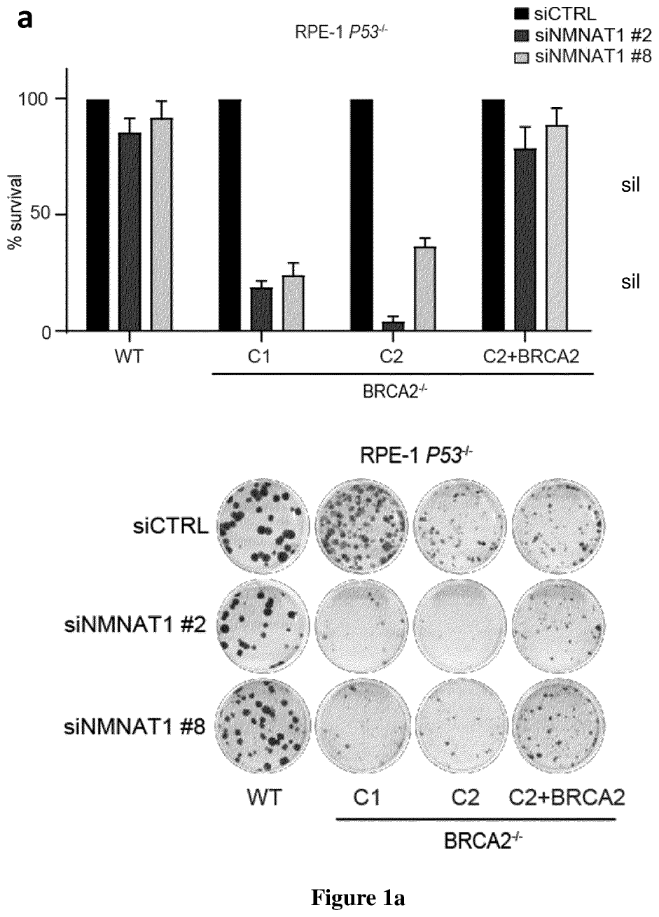

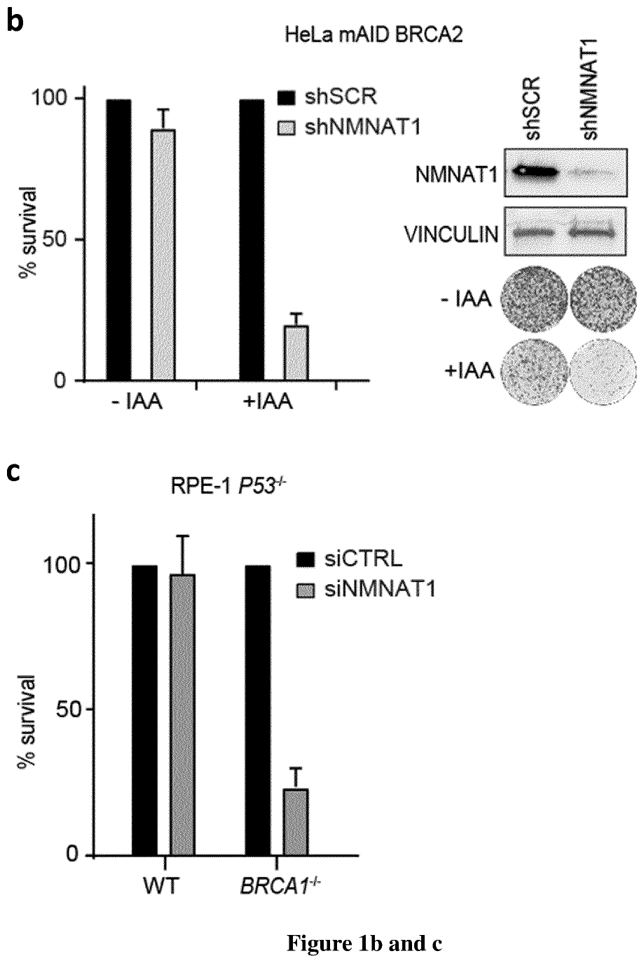

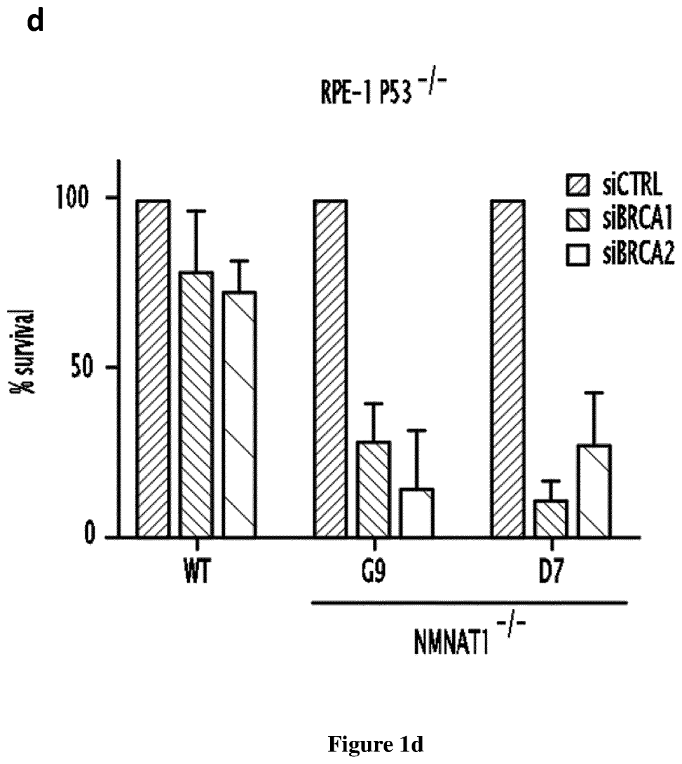

To study the role of NMNAT1-produced nuclear NAD+ in homologous recombination-deficient (HRD) cells, we generated several BRCA1/2 isogenic cell systems, namely BRCA1^{-/-} and BRCA2^{-/-} knockout clones in P53^{-/-} RPE-1 cells and BRCA2 mini auxin-inducible degron (mAID) in Hela cells. In P53^{-/-} RPE-1 cells, NMNAT1 knockdown by two different short interphering RNA sequences (siRNA) impaired the clonogenic ability of BRCA2^{-/-} clones, while having no effect on the survival of parental HRP cells (FIG. 1a). Complementation of BRCA2^{-/-} cells with a full length BRCA2 cDNA (C2+BRCA2) rescued the survival of BRCA2^{-/-} cells upon NMNAT1 depletion (FIG. 1a). Likewise, in the mAID BRCA2 HeLa isogenic model NMNAT1 knockdown by short hairpin RNA (shRNA) reduced cell growth only in the presence of auxin, i.e. upon BRCA2 protein degradation (FIG. 1b). Furthermore, NMNAT1 silencing also impaired the clonogenic ability of BRCA1^{-/-} P53^{-/-} RPE-1 cells, while sparing parental HRP cells (FIG. 1c). These data suggest that NMNAT1 is synthetically lethal with BRCA1/2.

To corroborate these findings, we used a complementary approach by silencing BRCA1/2 in NMNAT1^{-/-} RPE-1 cells that were generated by CRISP-Cas9 genome editing. While having only a mild effect on P53^{-/-} RPE-1 cells, knockdown of BRCA1/2 by siRNA completely impaired the clonogenic ability of NMNAT1^{-/-} P53^{-/-} RPE-1 cells (FIG. 1d).

Next, we compared siNMNAT1 cytotoxicity between HRP and HR-deficient (HRD) cancer cells. siNMNAT1 had a strong impact on the clonogenic ability of HRD cells, whereas it induced only mild effects in HRP cells, including the immortalized normal breast epithelial MCF-10A cells (FIG. 1e). Altogether, we collected data showing that NMNAT1 is synthetically lethal with BRCA1/2.

Considering that HRD cells have higher level of replication stress than HRP cells, we reasoned that NMNAT1 might play a key role in maintenance of genome stability, which would explain why its loss is detrimental only in BRCA-deficient cells. To test this, we quantified DNA breaks in RPE-1 cells with and without NMNAT1 by alkaline COMET assay. P53^{-/-} NMNAT1^{-/-} clones showed higher amount of DNA lesions if compared with parental cells (FIG. 1f), indicating that NMNAT1 contributes to maintain genome stability.

Accumulation of DNA lesions can result in increased sensitivity to genotoxic drugs. Given that NMNAT1 functions upstream of PARP1 by providing NAD⁺, the reduced PARP1 activity upon NMNAT1 loss might account for the observed synthetic lethality between NMNAT1 and BRCA. Interestingly, NMNAT1^{-/-} cells were sensitized to the PARP inhibitor rucaparib (PARPi) (FIG. 1g), suggesting that the role of NMNAT1 is not completely epistatic with PARP1, but rather nuclear NAD⁺ could be used by other downstream enzymes to sustain HRD cell survival.

### NMNAT1 Loss is not Detrimental in Normal Immortalized P53+/+ Cells

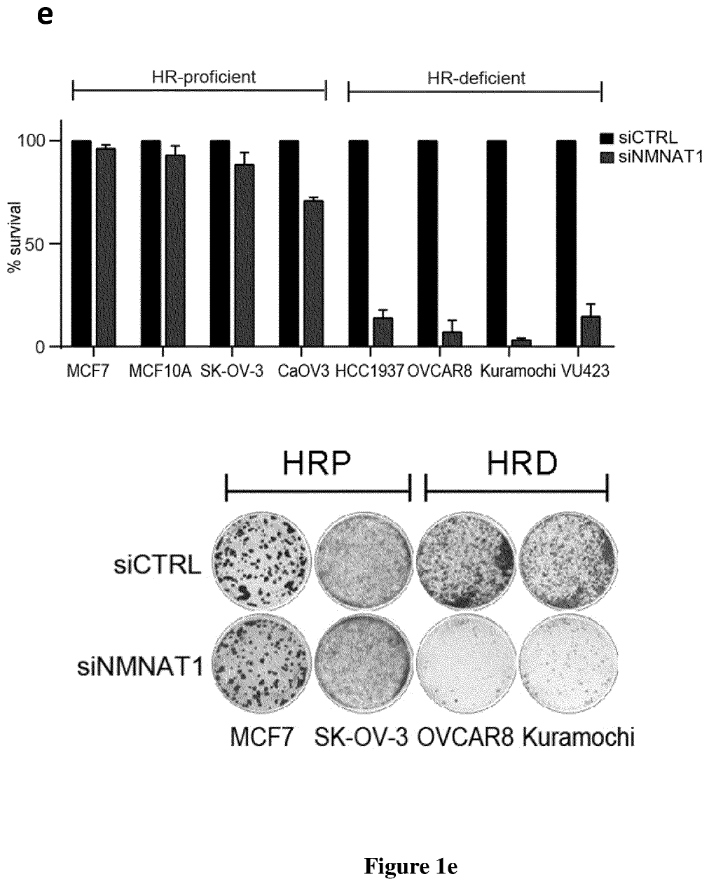

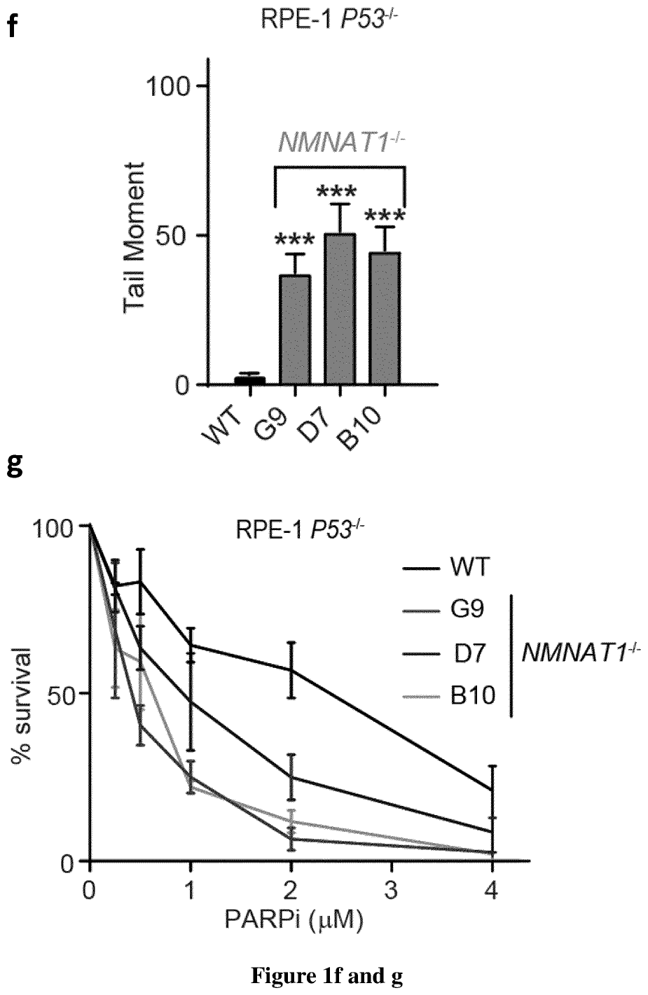

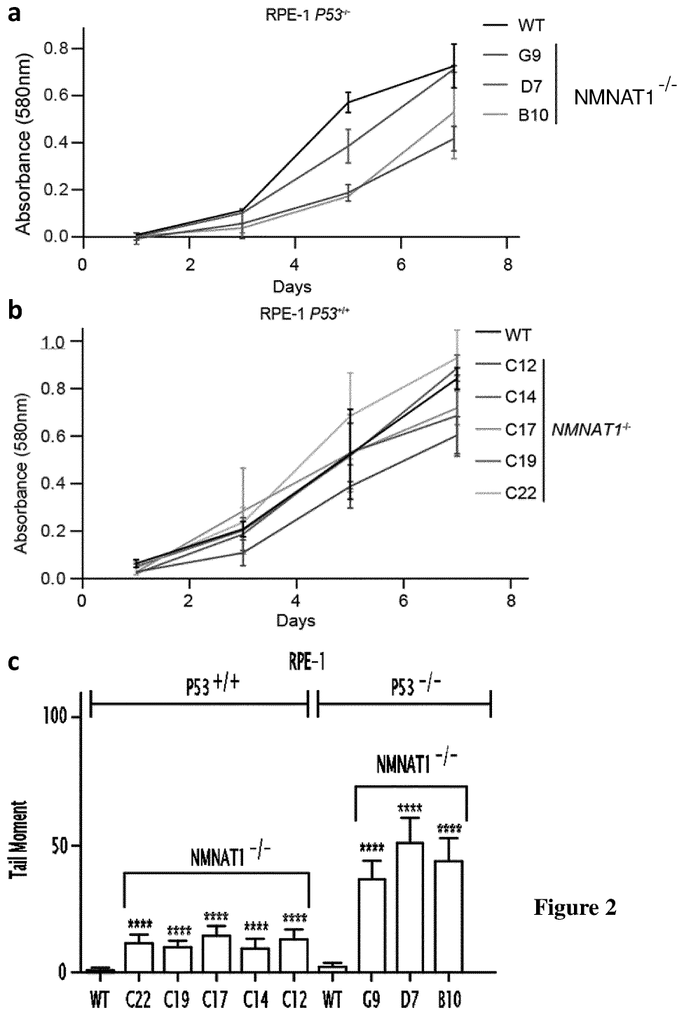

For the successful translation of synthetic lethal interactions into cancer treatments, one important consideration is the toxic impact of the inhibition of the gene under investigation in normal tissue. To rule out any possible detrimental effect of NMNAT1 loss in HRP cells, we monitored the proliferation rate of P53^{-/-} NMNAT1^{-/-} RPE-1 and parental cells for one week. All the three NMNAT1^{-/-} clones showed a mild reduction in terms of proliferation when compared to parental P53^{-/-} RPE-1 cells (FIG. 2a). To better evaluate the effect of NMNAT1 loss in normal tissues, we generated NMNAT1 knockout clones in P53^{+/+} RPE-1, as normal cells are P53-proficient. Contrarily, when P53^{+/+} NMNAT1^{-/-} RPE-1 clones were compared to parental cells, we observed that NMNAT1 loss did not have any impact in terms of cell proliferation, as shown by the similar growth of P53^{+/+} NMNAT1-clones (C12, C14, C17 C19 and C22) and parental P53^{+/+} cells (FIG. 2b). These data indicate that whereas NMNAT1 loss slightly reduces the proliferation of P53^{-/-} cells, it does not have any detrimental effect in P53^{+/+} cells, thus suggesting that targeting NMNAT1 could have no toxic impact in normal tissue.

The observed accumulation of DNA lesions in NMNAT1^{-/-} P53^{-/-} cells (FIG. 1g) might account for the reduced proliferation upon NMNAT1 loss in P53^{-/-} RPE-1 cells (FIG. 2a). However, when we measured DNA breaks by alkaline COMET assay in NMNAT1^{-/-} P53^{+/+} cells, the increase of DNA breaks was less considerable than in NMNAT1^{-/-} P53^{-/-} RPE-1 if compared to the respective parental cells (FIG. 2c). These results are consistent with the observation that NMNAT1 loss had no impact on the proliferation of NMNAT1^{-/-} P53^{+/+} cells and thus does not have any detrimental effect on normal cells.

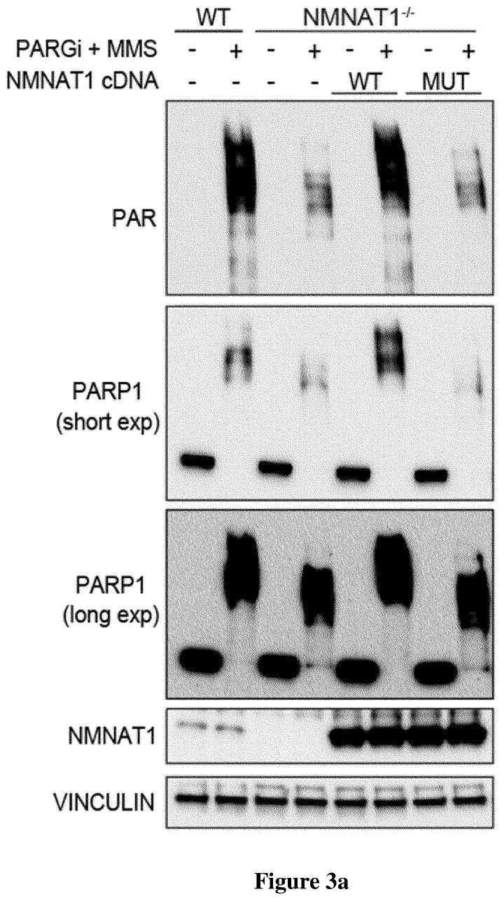

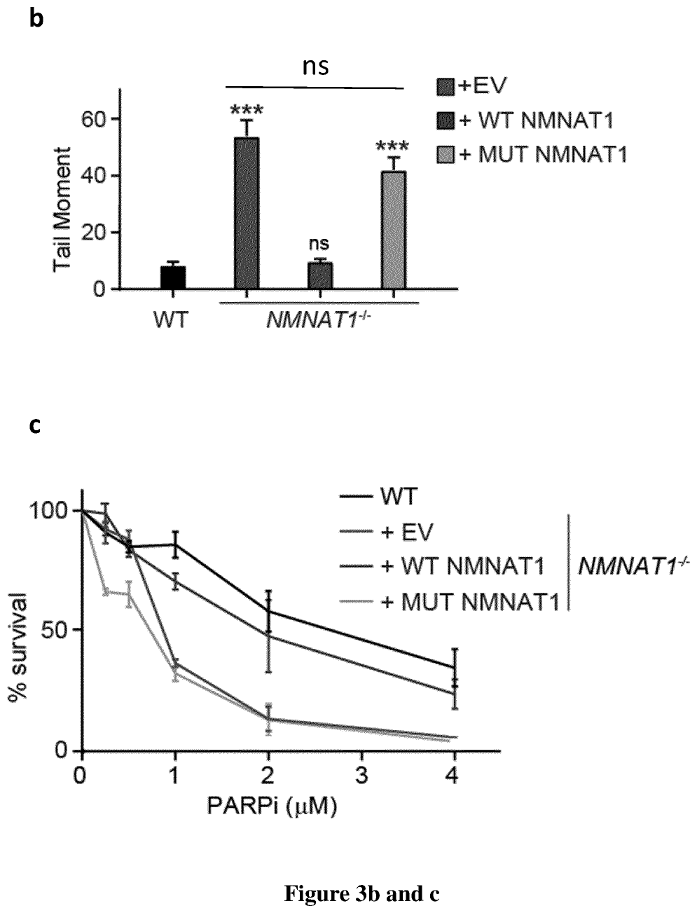

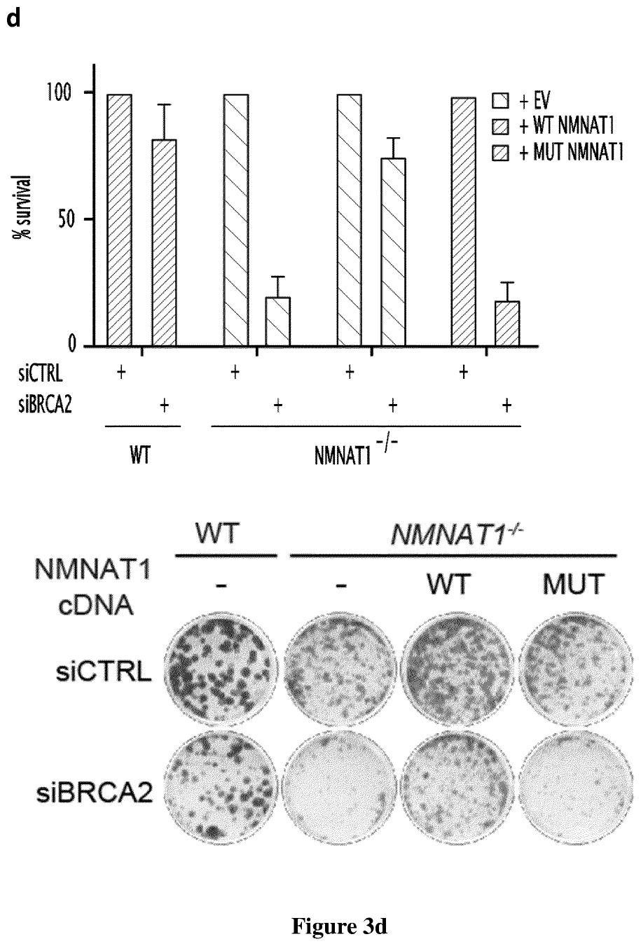

To evaluate whether the catalytic activity of NMNAT1, i.e. nuclear NAD⁺, is essential for the survival of HRD cells, we complemented NMNAT1^{-/-} RPE-1 cells with either wild type (WT) or a catalytically-dead version of the enzyme (W169A, MUT) and tested cell survival upon BRCA2 knockdown. While complementation with WT NMNAT1 increased PARP1 activation, and thus was able to rescue the amount of DNA lesions in NMNAT1^{-/-} cells to the same extent than in parental cells, the W169A mutant did not (FIG. 3a,b). In accordance with these results, both PARPi sensitivity and synthetic lethality with BRCA2 were rescued by WT NMNAT1 complementation, but not by W169A NMNAT1 (FIG. 3c,d). Altogether, these datas demonstrate that NMNAT1, a nuclear enzyme other than PARP1, is crucial for the survival of HRD cells. These results indicate that NMNAT1 is a key factor which activities are necessary for the survival of HRD cells.

Inhibition of NMNAT1 Kills PARP-Inhibitor and Cisplatin-Resistant BRCA1/2-Mutated Tumors, Including Those with Somatic Reversion of the BRCA1/2 Mutations

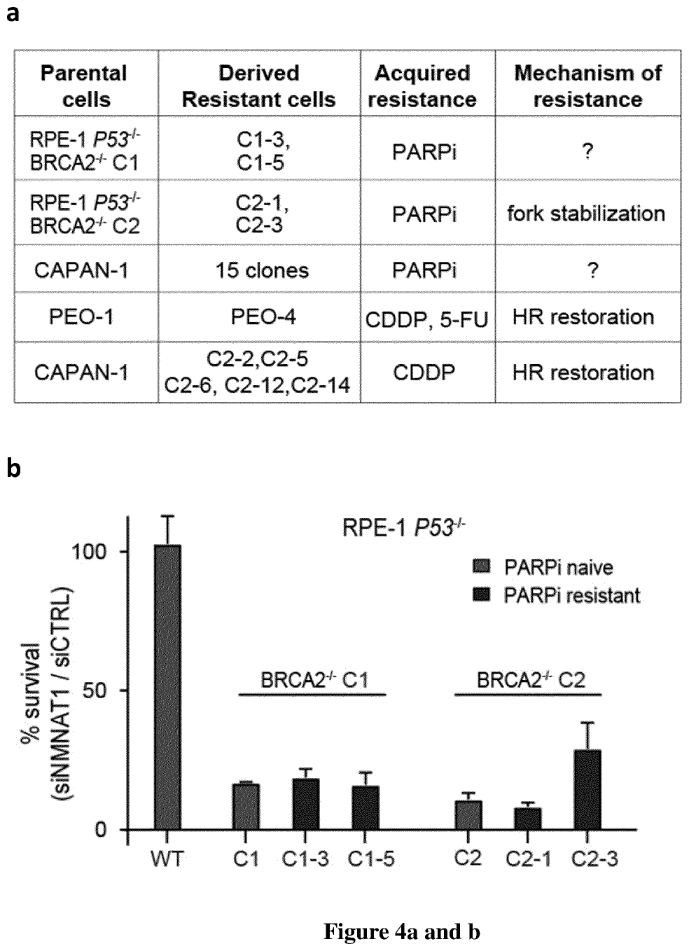

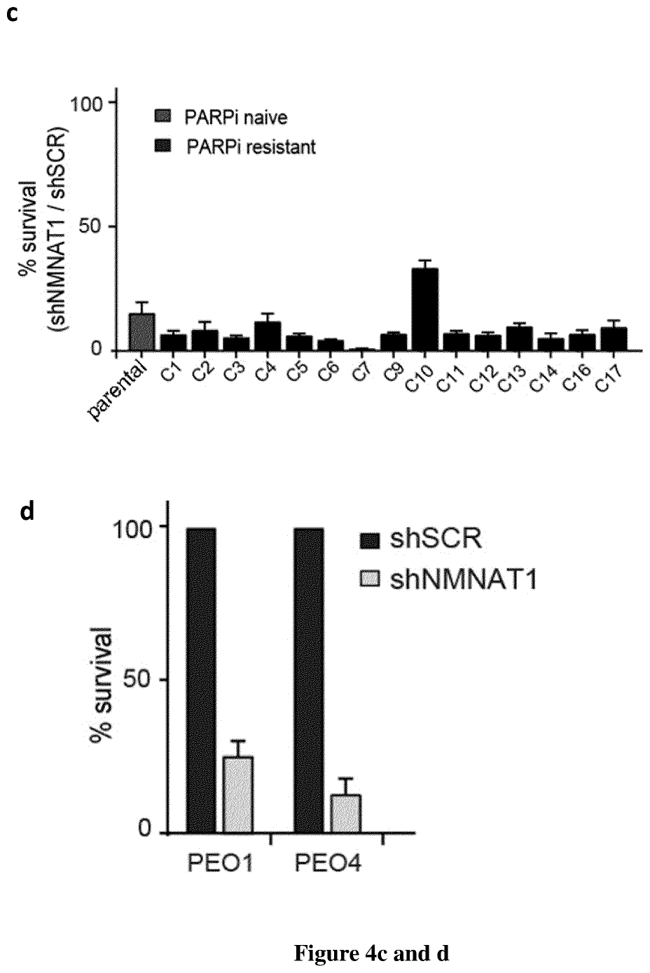

Despite the striking cytotoxic effect of PARPi in BRCA-mutated cells, insurgence of resistance is ubiquitous in clinic and calls for the design of alternative therapies for the treatment of advanced diseases. Our findings that inhibition of NMNAT1 kills HRD cells in a PARP1-independent manner suggest that targeting this axis might also tackle BRCA-mutated cells that developed resistance to PARPi. To test this, the inventors used several cellular models, which recapitulated the major known mechanisms of resistance, including fork stabilization and HR restoration (FIG. 4a).

First, the inventors generated resistant cells by continuous exposure of BRCA2^{-/-} RPE-1 to rucaparib. The inventors did not observe HR restoration in any of the derived clones, but resistance arose through fork stabilization. In those clones, NMNAT1 knockdown by siRNA impaired the clonogenic ability to a similar extent than that of the drug-naïve BRCA2^{-/-} cells, while sparing the HRP RPE-1 (FIG. 4b).

Then using a similar approach, several clones were derived from the BRCA2-mutated pancreatic cancer cell line CAPAN-1. After becoming resistant to rucaparib, those clones did not restore HR but rather developed resistance through other mechanisms that need to be further investigated. Knockdown of NMNAT1 impaired the clonogenic ability of all the CAPAN-1-derived resistant clones (FIG. 4c). Together, these data show that targeting NMNAT1 also kills PARPi-resistant HRD cells.

Nonetheless, HR restoration by secondary mutations in the BRCA genes is the only mechanism of resistance to PARPi validated so far in clinic. For this reason, the inventors evaluated the effect of NMNAT1 inhibition in HRD cells that developed chemo-resistance through BRCA2 secondary mutations restoring the open reading frame of the gene and thus HR. In particular, the inventors tested the chemo-resistant HR-restored ovarian cancer cell line PEO4—together with its BRCA2-mutated paired parental PEO1 cells- and five clones derived from prolonged in vitro cisplatin exposure of CAPAN-1 cells, each bearing different secondary mutation in BRCA2 gene. Surprisingly, shRNA-mediated knockdown of NMNAT1 impaired the colony formation ability of both PEO4 cells (FIG. 4d) and resistant CAPAN-1 clones, suggesting that targeting NMNAT1 kills chemo-resistant cells regardless the mechanism of drug resistance.

### NMNAT1 Inhibition Kills Homologous Recombination (HR)-Deficient (HRD) Tumor Cells In Vivo

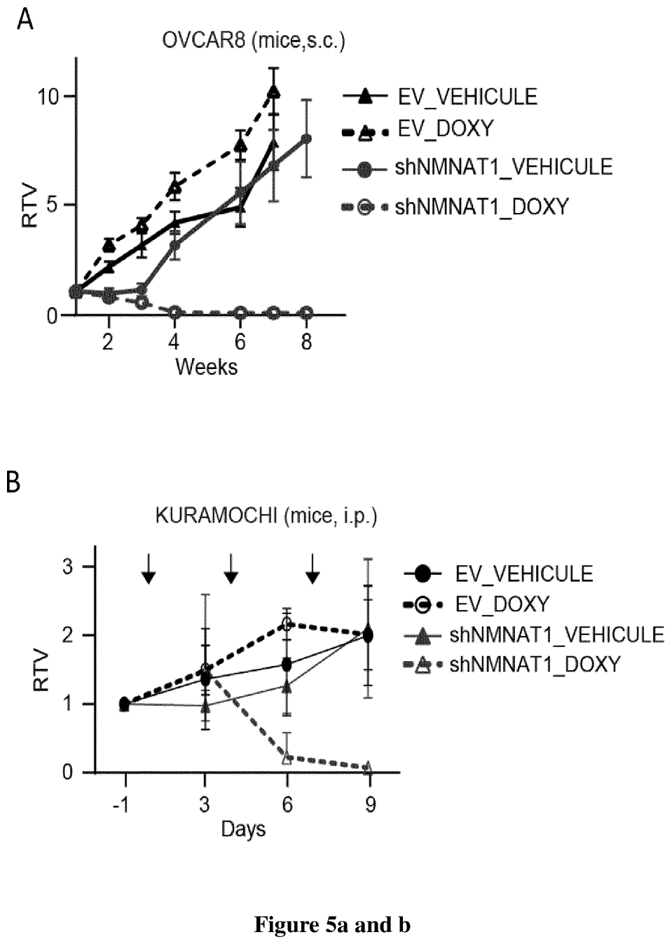

The inventors performed in vivo xenotransplantation studies in mice, using HRD human ovarian tumor lines expressing either doxycycline-inducible NMNAT1 or scrambled (Scr) shRNA. NMNAT1 depletion significantly impaired BRCA1-deficient (OVCAR8) xenograft tumor growth, in athymic nude mice (FIG. 5A). Next, the inventors xenotransplanted the luciferase-tagged Kuramochi (BRCA2-deficient) line intraperitoneally in NOD scid gamma (NSG) mice and waited 3 months so the tumors burden reached high radiance signal (i.e. confluency in the peritoneum). Strikingly, NMNAT1 depletion in these conditions achieved rapid and significant Kuramochi (BRCA2-deficient) tumor killing (FIG. 5B).

These data demonstrate that HRD cells are highly sensitive to NMAT1 inhibition in vivo.

### NMNAT1 Inhibition Kills PARPi-Resistant HRD Tumor Cells In Vivo

The inventors collected data in vitro showing that targeting NMNAT1 kills chemoresistant HRD cells regardless the mechanism of acquired resistance.

To translate these findings into a translational setting, the inventors derived a PARPi-resistant clone from the PARPi-naive BRCA1-deficient OVCAR8 lines, expressed either doxycycline-inducible NMNAT1 or scrambled (Scr) shRNA in these cells, and xenograft them in athymic nude mice.

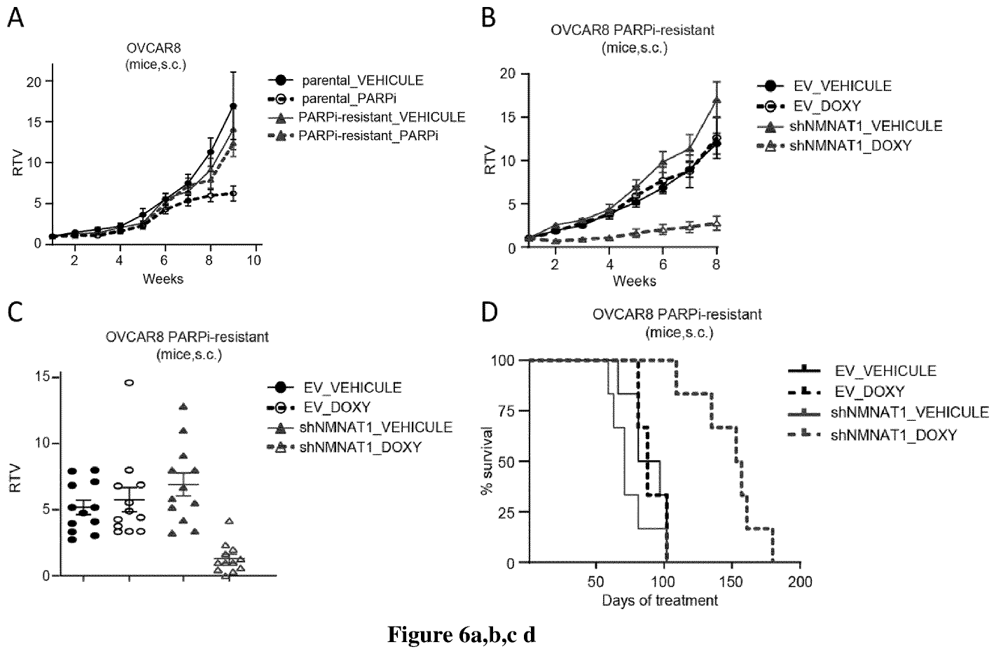

As expected the OVCAR8 PARPi-resistant cells were resistant to the PARPi rucaparib in vivo (FIG. 6A). It was found that NMNAT1 depletion significantly impaired tumor growth (FIG. 6B, C). Moreover, mice bearing NMNAT1-depleted tumors had a survival advantage compared to control mice (FIG. 6D).

### REFERENCES

Throughout this application, various references describe the state of the art to which this invention pertains. The disclosures of these references are hereby incorporated by reference into the present disclosure.
Bryant, H. E.; Schultz, N.; Thomas, H. D.; Parker, K. M.; Flower, D.; Lopez, E.; Kyle, S.; Meuth, M.; Curtin, N. J.; Helleday, T., Specific killing of BRCA2-deficient tumours with inhibitors of poly(ADP-ribose) polymerase. 2005, Nature.
Farmer, H.; McCabe, N.; Lord, C. J.; Tutt, A. N.; Johnson, D. A.; Richardson, T. B.; Santarosa, M.; Dillon, K. J.; Hickson, I.; Knights, C.; Martin, N. M.; Jackson, S. P.; Smith, G. C.; Ashworth, A., Targeting the DNA repair defect in BRCA mutant cells as a therapeutic strategy. 2005, Nature.
Pujade-Lauraine, E.; Ledermann, J. A.; Selle, F.; Gebski, V.; Penson, R. T.; Oza, A. M.; Korach, J.; Huzarski, T.; Poveda, A.; Pignata, S.; Friedlander, M.; Colombo, N.; Harter, P.; Fujiwara, K.; Ray-Coquard, I.; Banerjee, S.; Liu, J.; Lowe, E. S.; Bloomfield, R.; Pautier, P., Olaparib tablets as maintenance therapy in patients with platinum-sensitive, relapsed ovarian cancer and a BRCA1/2 mutation (SOLO2/ENGOT-Ov21): a double-blind, randomised, placebo-controlled, phase 3 trial. 2017, The Lancet. Oncology.
Konstantinopoulos, P. A.; Ceccaldi, R.; Shapiro, G. I.; D'Andrea, A. D., Homologous Recombination Deficiency: Exploiting the Fundamental Vulnerability of Ovarian Cancer. 2015, Cancer discovery.
Cambronne, X. A.; Kraus, W. L., Location, Location, Location: Compartmentalization of NAD (+) Synthesis and Functions in Mammalian Cells. 2020, Trends in biochemical sciences.
Lau, C.; Niere, M.; Ziegler, M., The NMN/NaMN adenylyltransferase (NMNAT) protein family. 2009, Frontiers in bioscience.
Gibson, B. A.; Kraus, W. L., New insights into the molecular and cellular functions of poly(ADP-ribose) and PARPs. 2012, Nature reviews. Molecular cell biology.
Gupte, R.; Liu, Z.; Kraus, W. L., PARPs and ADP-ribosylation: recent advances linking molecular functions to biological outcomes. 2017, Genes & development.
Maya-Mendoza, A. et al. High speed of fork progression induces DNA replication stress and genomic instability. Nature 559, 279-284 (2018).
Hanzlikova, H. et al. The Importance of Poly(ADP-Ribose) Polymerase as a Sensor of Unligated Okazaki Fragments during DNA Replication. Molecular Cell 71, 319-331.e3 (2018).
Patel, P. S., Algounch, A. & Hakem, R. Exploiting synthetic lethality to target BRCA1/2-deficient tumors: where we stand. Oncogene 2021 40:17 40, 3001-3014 (2021).
Gogola, E., Rottenberg, S. & Jonkers, J. Resistance to PARP Inhibitors: Lessons from Preclinical Models of BRCA-Associated Cancer. https://doi.org/10.1146/annurev-cancerbio-030617-050232 3, 235-254 (2019).
Kusumanchi, P., Zhang, Y., Jani, M. et al. Nicotinamide mononucleotide adenylyltransferase2 overexpression enhances colorectal cancer cell-kill by Tiazofurin. Cancer Gene Ther 20, 403-412 (2013).

1.-11. (canceled)

12. A method for treating HRD cancer and BRCA-associated cancer comprising administering to a subject in need thereof, a therapeutically effective amount of a NMNAT1 inhibitor.

13. The method according to claim 12, wherein the cancer is chemo-resistance BRCA-associated cancer.

14. The method according to claim 12, wherein the cancer is PARPi resistant BRCA-associated cancer or cisplatin resistant BRCA-associated cancer, including those with somatic reversion of the BRCA mutation and HR restoration.

15. The method according to claim 12, wherein said NMNAT1 inhibitor is a small organic molecule, a polypeptide, an aptamer, an oligonucleotide or an antibody.

16. The method according to claim 12, wherein said NMNAT1 inhibitor is an antisense oligonucleotide, a siRNA, a shRNA, a DNA aptamer or a RNA aptamer.

17. The method according to claim 12, wherein said NMNAT1 inhibitor is administered in combination with a PARP inhibitor, optionally said PARP inhibitor is selected from the group consisting of olaparib, rucaparib, niraparib, talazoparib, iniparib, veliparib, Pamiparib (BGB-290), CEP 9722, E7016, E7449 and 3-Aminobenzamide.

18. The method according to claim 12, wherein said NMNAT1 inhibitor is administered in combination with cisplatin or a Pole inhibitor such as novobiocin.

19. The method according to claim 13, wherein said NMNAT1 inhibitor is a small organic molecule, a polypeptide, an aptamer, an oligonucleotide or an antibody.

20. The method according to claim 13, wherein said NMNAT1 inhibitor is an antisense oligonucleotide, a siRNA, a shRNA, a DNA aptamer or a RNA aptamer.

21. The method according to claim 13, wherein said NMNAT1 inhibitor is administered in combination with a PARP inhibitor, optionally said PARP inhibitor is selected from the group consisting of olaparib, rucaparib, niraparib, talazoparib, iniparib, veliparib, Pamiparib (BGB-290), CEP 9722, E7016, E7449 and 3-Aminobenzamide.

22. The method according to claim 13, wherein said NMNAT1 inhibitor is administered in combination with cisplatin or a Pole inhibitor such as novobiocin.

23. The method according to claim 14, wherein said NMNAT1 inhibitor is a small organic molecule, a polypeptide, an aptamer, an oligonucleotide or an antibody.

24. The method according to claim 14, wherein said NMNAT1 inhibitor is an antisense oligonucleotide, a siRNA, a shRNA, a DNA aptamer or a RNA aptamer.

25. The method according to claim 14, wherein said NMNAT1 inhibitor is administered in combination with a PARP inhibitor, optionally said PARP inhibitor is selected from the group consisting of olaparib, rucaparib, niraparib, talazoparib, iniparib, veliparib, Pamiparib (BGB-290), CEP 9722, E7016, E7449 and 3-Aminobenzamide.

26. The method according to claim 14, wherein said NMNAT1 inhibitor is administered in combination with cisplatin or a Pole inhibitor such as novobiocin.

27. A method for treating HRD cancer and BRCA-associated cancer comprising administering to a subject in need thereof, a pharmaceutical composition comprising a NMNAT1 inhibitor and a PARP inhibitor.

28. The method according to claim 27, wherein said NMNAT1 inhibitor is a small organic molecule, a polypeptide, an aptamer, an oligonucleotide or an antibody.

29. The method according to claim 27, wherein the PARP inhibitor selected from the group consisting of olaparib, rucaparib, niraparib, talazoparib, iniparib, veliparib, Pamiparib (BGB-290), CEP 9722, E7016, E7449 and 3-Aminobenzamide.

30. A method for treating HRD cancer and BRCA-associated cancer comprising administering to a subject in need thereof, a pharmaceutical composition comprising a NMNAT1 inhibitor and cisplatin or a Pole inhibitor such as novobiocin.
